(19) United States
(12) Patent Application Publication
(10) Pub. No.: US 2024/0300876 A1
(43) Pub. Date: Sep. 12, 2024

(54) METHODS OF PRODUCING LOW-ACETAL ETHANOL

(71) Applicant: SUPERBREWED FOOD, INC., New Castle, DE (US)

(72) Inventors: Bryan P. TRACY, Wilmington, DE (US); Dale A. MONCEAUX, Loveland, OH (US); Aharon M. Eyal, Jerusalem (IL)

(21) Appl. No.: 18/270,843

(22) PCT Filed: Jan. 7, 2022
(86) PCT No.: PCT/IB2022/050104

§ 371 Date: Jul. 4, 2023

Related U.S. Application Data
(60) Provisional application No. 63/134,604, filed on Jan. 7, 2021.

(51) Int. Cl. C07C 29/84; C07C 29/09; C07C 29/88; C12P 7/06

(52) U.S. Cl. CPC C07C 29/84; C07C 29/09; C07C 29/88; C12P 7/06

(57) ABSTRACT

Provided is a method for the production of refined, low-acetal ethanol, comprising providing a fermentation product produced by fermentation of a carbon source with an ethanol-producing organism, which fermentation product comprises between 30% wt and 99% wt ethanol, between 70% wt and 1% wt water and between 20 and 2000 parts per million acetal; contacting said fermentation product with an acid catalyst, whereby at least 20% of the acetal hydrolyses to form a hydrolysis product comprising ethanol, water, acetaldehyde and optionally residual acetal; and distilling said hydrolysis product, whereby distilled, low-acetal ethanol is formed.

### CROSS-REFERENCE TO RELATED APPLICATION

The present application gains priority from U.S. Provisional Application No. 63/134,604 filed Jan. 7, 2021 which is incorporated by reference as if fully set-forth herein.

### FIELD OF THE INVENTION

The present invention relates to methods of producing low-acetal ethanol and more specifically to methods comprising fermentation of a carbon source with an ethanol-producing organism to form a fermentation product; contacting the fermentation product with an acid catalyst to form a hydrolysis product; and distilling the hydrolysis product to form distilled, low-acetal ethanol.

### BACKGROUND

Acetal is a functional group with the general formula —R₁(R₂)C(—OR₃)—OR₄. Diethoxyethane (also referred to as diethyl acetal) is the simplest form of acetal, wherein R₁ and R₂ are both hydrogen atoms and R₃ and R₄ are both methyl groups.

During production of fuel-grade ethanol, a purification step (typically used to increase purity from 96% to at least 99% purity) is commonly carried out using a molecular sieve. Acetal which is formed on the molecular sieve is generally recycled as part of the sieve regeneration stream to a subsequent concentration step, such that the acetal is present in the final product.

There is an ongoing need for improved methods of producing low-acetal ethanol, for purposes such as hand-sanitizing and other medical uses.

### SUMMARY OF THE INVENTION

According to an aspect of some embodiments of the present invention, there is provided a method for the production of refined, low-acetal ethanol, comprising
(i) providing a feed stream comprising between 30% wt and 99% wt ethanol, between 70% wt and 1% wt water and between 20 and 2000 parts per million acetal;
(ii) contacting said feed stream with an acid catalyst, whereby at least 20% of the acetal hydrolyses to form a hydrolysis product comprising ethanol, water, acetaldehyde and optionally residual acetal; and
(iii) distilling said hydrolysis product, whereby refined, low-acetal ethanol is formed.

According to a further aspect of some embodiments of the present invention, there is provided a method for the production of refined, low-acetal ethanol, comprising
(i) providing a carbon source;
(ii) fermenting said carbon source to form a fermentation liquor;
(iii) concentrating at least a fraction of said fermentation liquor to form a concentrated ethanol solution containing between 85% wt and 96% wt ethanol and between 4% wt and 15% wt of water;
(iv) dewatering a first fraction of said concentrated ethanol solution on a molecular sieve to form a fuel-grade, dewatered ethanol solution containing less than 1% wt water, wherein said first fraction comprises between 10% wt and 95% wt of the ethanol in the concentrated ethanol solution;
(v) regenerating said molecular sieve, whereby a feed stream comprising a regeneration solution is formed, which feed stream comprises between 30% wt and 99% wt ethanol, between 70% wt and 1% wt water and between 20 and 2000 parts per million acetal;
(vi) contacting said feed stream with an acid catalyst, whereby at least 20% of the acetal hydrolyses to form a hydrolysis product comprising ethanol, water, acetaldehyde and optionally residual acetal;
(vii) recycling said hydrolysis product to said fermentation liquor prior to or during said concentrating of said fermentation liquor; and
   distilling a second fraction of said concentrated ethanol solution, comprising between 5% wt and 90% wt of the ethanol in the concentrated ethanol solution, whereby refined, low-acetal ethanol is formed.

According to a further aspect of some embodiments of the present invention, there is provided a method for the production of refined, low-acetal ethanol, comprising
(i) providing a carbon source;
(ii) fermenting said carbon source to form a fermentation liquor;
(iii) concentrating at least a fraction of said fermentation liquor to form a concentrated ethanol solution containing between 85% wt and 96% wt ethanol and between 4% wt and 15% wt of water;
(iv) dewatering a first fraction of said concentrated ethanol solution on a molecular sieve to form a fuel-grade, dewatered ethanol solution containing less than 1% wt water, wherein said first fraction comprises between 10% wt and 95% wt of the ethanol in the concentrated ethanol solution;
(v) regenerating said molecular sieve, whereby a regeneration solution is formed,
(vi) recycling said regeneration solution to said fermentation liquor prior to or during said concentrating of said fermentation liquor, whereby said feed stream is formed, which feed stream comprises between 30% wt and 99% wt ethanol, between 70% wt and 1% wt water and between 20 and 2000 parts per million acetal;
(vii) contacting a fraction of said feed stream comprising between 5% wt and 90% wt of the ethanol in the feed stream with an acid catalyst, whereby at least 20% of the acetal hydrolyses to form a hydrolysis product comprising ethanol, water, acetaldehyde and optionally residual acetal;
(viii) distilling at least a fraction of said hydrolysis product, whereby refined, low-acetal ethanol is formed.

According to a further aspect of some embodiments of the present invention, there is provided a method for the production of refined, low-acetal ethanol, comprising
(i) providing a carbon source;
(ii) fermenting said carbon source to form a fermentation liquor;
(iii) concentrating at least a fraction of said fermentation liquor to form a concentrated ethanol solution containing between 85% wt and 96% wt ethanol and between 4% wt and 15% wt of water;
(iv) dewatering a first fraction of said concentrated ethanol solution on a molecular sieve to form a fuel-grade, dewatered ethanol solution containing less than 1% wt water, wherein said first fraction comprises between 10% wt and 95% wt of the ethanol in the concentrated ethanol solution;
(v) regenerating said molecular sieve, whereby a regeneration solution is formed,
(vi) recycling said regeneration solution to said fermentation liquor prior to or during said concentrating of said fermentation liquor;
(vii) distilling a second fraction of said concentrated ethanol solution comprising between 5% wt and go % wt of the ethanol in said concentrated solution, whereby a feed stream comprising a fraction of the distilled ethanol is formed, which feed stream comprises between 30% wt and 99% wt ethanol, between 70% wt and 1% wt water and between 20 and 2000 parts per million acetal;
(viii) contacting at least a fraction of said feed stream comprising between 5% wt and 90% wt of the ethanol in the feed stream with an acid catalyst, whereby at least 20% of the acetal hydrolyses to form a hydrolysis product comprising ethanol, water, acetaldehyde and optionally residual acetal; and
   recycling said hydrolysis product to said distilling and/or distilling said hydrolysis product to a second distilling.

According to an embodiment, said acid catalyst is selected from the group consisting of mineral acids, polymers carrying —SO₃ functions and acidic zeolites.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 is a schematic representation of a method in accordance with an embodiment of the present invention;

FIG. 2 is a schematic representation of a method in accordance with an alternative embodiment of the present invention;

FIG. 3 is a schematic representation of a method in accordance with a further alternative embodiment of the present invention; and

FIG. 4 is a schematic representation of a method in accordance with a further alternative embodiment of the present invention.

### DETAILED DESCRIPTION OF THE INVENTION

Acetal is formed in a two-step reaction, both steps being catalyzed by acid catalysts. First, acetaldehyde reacts with one ethanol molecule to form hemiacetal. In the next step, a second ethanol molecule binds to the hemiacetal and a water molecule is released. The system therefore involves the following equilibrium reaction:

Acetaldehyde+ethanol ↔hemiacetal+ethanol ↔acetal+water

It is known that 190 and 200 proof ethanol contains acetaldehyde, hemiacetal and acetal. The reaction is driven forward in the direction of hemiacetal and acetal production upon removal of water (resulting in increased ethanol concentration), which can be achieved with high efficiency by use of a molecular sieve.

According to an aspect of some embodiments of the present invention, there is provided a method for the production of refined, low-acetal ethanol, comprising
(i) providing a feed stream comprising between 30% wt and 99% wt ethanol (such as at least 30% wt, about 35% wt, about 40% wt, about 45% wt, about 50% wt, about 55% wt, about 60% wt, about 65% wt, about 70% wt, about 75% wt, about 80% wt, about 85% wt, about 90% wt or up to 99% wt ethanol), between 70% wt and 1% wt water (such as at least 1% wt, about 5% wt, about 10% wt, about 15% wt, about 20% wt, about 25% wt, about 30% wt, about 35% wt, about 40% wt, about 45% wt, about 50% wt, about 55% wt, about 60% wt, about 65% wt or up to 70% wt water) and between 20 and 2000 parts per million acetal (such as at least 20 ppm, about 40 ppm, about 60 ppm, about 80 ppm, about 100 ppm, about 200 ppm, about 400 ppm, about 600 ppm, about 800 ppm, about 1000 ppm, about 1200 ppm, about 1400 ppm, about 1600 ppm, about 1800 ppm or up to 2000 ppm acetal);
(ii) contacting said feed stream with an acid catalyst, whereby at least 20% of the acetal (such as at least 20%, at least 30%, at least 40%, at least 50%, at least 60%, at least 70%, at least 80%, or at least 90% of the acetal) hydrolyses to form a hydrolysis product comprising ethanol, water, acetaldehyde and optionally residual acetal; and
(iii) distilling said hydrolysis product, whereby refined, low-acetal ethanol is formed.

According to a further aspect of some embodiments of the present invention, there is provided a method for the production of refined, low-acetal ethanol, comprising
(i) providing a carbon source;
(ii) fermenting said carbon source to form a fermentation liquor;
(iii) concentrating at least a fraction of said fermentation liquor to form a concentrated ethanol solution containing between 85% wt and 96% wt ethanol (such as at least 85% wt, about 86% wt, about 87% wt, about 88% wt, about 89% wt, about go % wt, about 91% wt, about 92% wt, about 93% wt, about 94% wt, about 95% wt or up to 96% wt ethanol) and between 4% wt and 15% wt of water (such as at least 4% wt, about 5% wt, about 6% wt, about 7% wt, about 8% wt, about 9% wt, about 10% wt, about 11% wt, about 12% At, about 13% wt, about 14% wt or up to 15% wt water);
(iv) dewatering a first fraction of said concentrated ethanol solution on a molecular sieve to form a fuel-grade, dewatered ethanol solution containing less than 1% wt water (such as less than 1% wt, less than 0.9% wt, less than 0.8% wt, less than 0.7% wt, less than 0.6% wt, less than 0.5% wt, less than 0.4% wt, less than 0.3% wt, less than 0.2% wt or even less than 0.1% wt water), wherein said first fraction comprises between 10% wt and 95% wt of the ethanol (such as at least 10% wt, about 15% wt, about 20% wt, about 25% wt, about 30% wt, about 35% wt, about 40% wt, about 45% wt, about 50% wt, about 55% wt, about 60% wt, about 65% wt, about 70% wt, about 75% wt, about 80% wt, about 90% wt or up to 95% wt of the ethanol) in the concentrated ethanol solution;
(v) regenerating said molecular sieve, whereby a feed stream comprising a regeneration solution is formed, which feed stream comprises between 3)% wt and 99% wt ethanol (such as at least 30% wt, about 35% wt, about 40% wt, about 45% wt, about 50% wt, about 55% wt, about 60% wt, about 65% wt, about 70% wt, about 75% wt, about 80% wt, about 85% wt, about 90% wt or up to 99% wt ethanol), between 70% wt and 1% wt water (such as at least 1% wt, about 5% wt, about 10% wt, about 15% wt, about 20% wt, about 25% wt, about 30% wt, about 35% wt, about 40% wt, about 45% wt, about 50% wt, about 55% wt, about 60% wt, about 65% wt or up to 70% wt water) and between 20 and 2000 parts per million acetal (such as at least 20 ppm, about 40 ppm, about 60 ppm, about 80 ppm, about 100 ppm, about 200 ppm, about 400 ppm, about 600 ppm, about 800 ppm, about 1000 ppm, about 1200 ppm, about 1400 ppm, about 1600 ppm, about 1800 ppm or up to 2000 ppm acetal);
(vi) contacting said feed stream with an acid catalyst, whereby at least 20% of the acetal (such as at least 20%, at least 30%, at least 40%, at least 50%, at least 60%, at least 70%, at least 80%, or at least 90% of the acetal) hydrolyses to form a hydrolysis product comprising ethanol, water, acetaldehyde and optionally residual acetal;
(vii) recycling said hydrolysis product to said fermentation liquor prior to or during said concentrating of said fermentation liquor; and
   distilling a second fraction of said concentrated ethanol solution, comprising between 5% wt and 90% wt of the ethanol such as at least 5% wt, about 10% wt, about 15% wt, about 20% wt, about 25% wt, about 30% wt, about 35% wt, about 40% wt, about 45% wt, about 50% wt, about 55% wt, about 60% wt, about 65% wt, about 70% wt, about 75% wt, about 80% wt, or up to 90% wt of the ethanol) in the concentrated ethanol solution, whereby refined, low-acetal ethanol is formed.

According to a further aspect of some embodiments of the present invention, there is provided a method for the production of refined, low-acetal ethanol, comprising
(i) providing a carbon source;
(ii) fermenting said carbon source to form a fermentation liquor;
(iii) concentrating at least a fraction of said fermentation liquor to form a concentrated ethanol solution containing between 85% wt and 96% wt ethanol and between 4% wt and 15% wt of water;
(iv) dewatering a first fraction of said concentrated ethanol solution on a molecular sieve to form a fuel-grade, dewatered ethanol solution containing less than 1% wt water, wherein said first fraction comprises between 10% wt and 95% wt of the ethanol in the concentrated ethanol solution;
(v) regenerating said molecular sieve, whereby a regeneration solution is formed,
(vi) recycling said regeneration solution to said fermentation liquor prior to or during said concentrating of said fermentation liquor, whereby said feed stream is formed, which feed stream comprises between 30% wt and 99% wt ethanol, between 70% wt and 1% wt water and between 20 and 2000 parts per million acetal;
(vii) contacting a fraction of said feed stream comprising between 5% wt and 90% wt of the ethanol in the feed stream with an acid catalyst, whereby at least 20% of the acetal hydrolyses to form a hydrolysis product comprising ethanol, water, acetaldehyde and optionally residual acetal;
(viii) distilling at least a fraction of said hydrolysis product, whereby refined, low-acetal ethanol is formed.

According to a further aspect of some embodiments of the present invention, there is provided a method for the production of refined, low-acetal ethanol, comprising
(i) providing a carbon source;
(ii) fermenting said carbon source to form a fermentation liquor;
(iii) concentrating at least a fraction of said fermentation liquor to form a concentrated ethanol solution containing between 85% wt and 96% wt ethanol and between 4% wt and 15% wt of water;
(iv) dewatering a first fraction of said concentrated ethanol solution on a molecular sieve to form a fuel-grade, dewatered ethanol solution containing less than 1% wt water, wherein said first fraction comprises between 10% wt and 95% wt of the ethanol in the concentrated ethanol solution;
(v) regenerating said molecular sieve, whereby a regeneration solution is formed,
(vi) recycling said regeneration solution to said fermentation liquor prior to or during said concentrating of said fermentation liquor;
(vii) distilling a second fraction of said concentrated ethanol solution comprising between 5% wt and 90% wt of the ethanol in said concentrated solution, whereby a feed stream comprising a fraction of the distilled ethanol is formed, which feed stream comprises between 30% wt and 99% wt ethanol, between 70% wt and 1% wt water and between 20 and 2000 parts per million acetal;
(viii) contacting at least a fraction of said feed stream comprising between 5% wt and go % wt of the ethanol in the feed stream with an acid catalyst, whereby at least 20% of the acetal hydrolyses to form a hydrolysis product comprising ethanol, water, acetaldehyde and optionally residual acetal; and
   recycling said hydrolysis product to said distilling and/or distilling said hydrolysis product to a second distilling.

According to an embodiment, said acid catalyst is selected from the group consisting of mineral acids, polymers carrying —SO₃ functions and acidic zeolites.

According to an embodiment, the carbon source is selected from the group consisting of starch, dextrose, maltodextrin, glucose, liquefied corn mash, fructose, xylose, glycerol, sucrose, hemicellulose, cellulose and combinations thereof.

According to an embodiment, the ethanol-producing organism is a yeast.

According to an embodiment, the fermentation product comprises at least 30% wt, about 40% wt, about 45% wt, about 50% wt, about 55% wt, about 60% wt, about 65% wt, about 70% wt, about 75% wt, about 80% wt, about 85% wt, about 90% wt, about 95% wt or up to 99% wt ethanol.

According to an embodiment, the fermentation product comprises at least 1% wt, about 5% wt, about 1)% wt, about 15% wt, about 20% wt, about 25% wt, about 30% wt, about 35% wt, about 40% wt, about 45% wt, about 50% wt, about 55% wt, about 60% wt, about 65% wt or up to 70% wt water.

According to an embodiment, the fermentation product comprises at least 20 ppm, about 40 ppm, about 60 ppm, about 80 ppm, about 100 ppm, about 200 ppm, about 400 ppm, about 600 ppm, about 800 ppm, about 1000 ppm, about 1200 ppm, about 1400 ppm, about 1600 ppm, about 1800 ppm or up to 2000 ppm acetal.

According to an embodiment, at least 20%, at least 30%, at least 40%, at least 50%, at least 60%, at least 70%, at least 80%, or at least 90% of the acetal hydrolyses to form a hydrolysis product.

According to an embodiment, providing said fermentation product comprises providing a carbon source, fermenting said carbon source to form a fermentation liquor and concentrating up said fermentation liquor to provide a concentrated fermentation liquor comprising between 85% wt and 96% wt ethanol (such as 85% wt, 86% wt, 87% wt, 88% wt, 89% wt, 90% wt, 91% wt, 92% wt, 93% wt, 94% wt, 95% wt or 96% wt ethanol), and between 15% wt and 4% wt water (such as 4% wt, 5% wt, 6% wt, 7% wt, 8% wt, 9% wt, 10% wt, n % wt, 12% wt, 13% wt, 14% wt or 15% wt).

According to an embodiment, said providing a carbon source comprises milling corn by a process selected from the group consisting of wet milling, dry milling or a combination thereof.

According to an embodiment, at least one selected from the group consisting of said fermentation liquor, said concentrated fermentation liquor and said feed stream further comprises at least one lower boiling point impurity having a boiling point at atmospheric pressure of less than 78° C. and/or at least one higher boiling point impurity having a boiling point of greater than 78° C. According to an embodiment, said concentrating is conducted in at least two steps (such as two steps, three steps, four steps or five steps), wherein a first of said at least two steps forms a partially concentrated fermentation liquor and a second of said at least two steps forms said concentrated fermentation liquor.

According to an embodiment, said distilling said hydrolysis product is conducted in at least two steps (such as two steps, three steps, four steps or five steps), wherein a first of said at least two steps removes acetaldehyde and optionally light impurities and a second of said at least two steps removes heavy impurities. According to an alternative embodiment, said distilling said hydrolysis product is conducted in at least two steps (such as two steps, three steps, four steps or five steps), wherein a first of said at least two steps removes heavy impurities and a second of said at least two steps removes acetaldehyde and optionally light impurities.

According to an embodiment, the method further comprises dewatering between 10% wt and 95% wt (such as at least 10% wt, about 15% wt, about 20% wt, about 25% wt, about 30% wt, about 30% wt, about 40% wt, about 45% wt, about 50% wt, about 55% wt, about 60% wt, about 65% wt, about 70% wt, about 75% wt, about 80% wt, about 90% wt or up to 95% wt) of said concentrated ethanol solution on a molecular sieve, wherein water is selectively adsorbed on said molecular sieve to form a fuel-grade, dewatered ethanol solution containing less than 1% wt water.

According to an embodiment, the method further comprises regenerating said molecular sieve whereby said water adsorbed on said molecular sieve is desorbed from said molecular sieve and a fermentation product comprising a regeneration solution is formed, wherein said fermentation product comprises between 30% wt and 99% wt ethanol (such as at least 30% wt, about 35% wt, about 40% wt, about 45% wt, about 50% wt, about 55% wt, about 60% wt, about 65% wt, about 70% wt, about 75% wt, about 80% wt, about 85% wt, about 90% wt or up to 99% wt ethanol), between 70% wt and 1% wt water (such as at least 1% wt, about 5% wt, about 10% wt, about 15% wt, about 20% wt, about 25% wt, about 30% wt, about 35% wt, about 40% wt, about 45% wt, about 50% wt, about 55% wt, about 60% wt, about 65% wt or up to 70% wt water) and between 20 and 2000 parts per million acetal (such as at least 20 ppm, about 40 ppm, about 60 ppm, about 80 ppm, about 100 ppm, about 200 ppm, about 400 ppm, about 600 ppm, about 800 ppm, about 1000 ppm, about 1200 ppm, about 1400 ppm, about 1600 ppm, about 1800 ppm or up to 2000 ppm acetal).

According to an embodiment, the method further comprises contacting said fermentation product comprising said regeneration solution with said acid catalyst and recycling said formed hydrolysis product to said fermentation liquor prior to said concentrating up or during said concentrating up of said fermentation product.

According to an embodiment, said acid catalyst is selected from the group consisting of mineral acids (such as nitric acid, sulfuric acid and the like), strong acid cation exchangers, polymers carrying —SO₃ functions and acidic zeolites.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “low-acetal ethanol” refers to ethanol having an acetal concentration of less than about 100 ppm.

As used herein, the term “wet milling” refers to a milling process in which corn is soaked or steeped in a liquid, such as water, prior to milling. As used herein, the term “dry milling” refers to a milling process which is devoid of an initial soaking or steeping step.

As used herein, the term “partially concentrated” with regard to a fermentation liquor refers to a fermentation liquor which comprises from about 30% wt to about 70 wt % water.

As used herein, the term “molecular sieve” refers to a material comprising pores of uniform size, wherein the size of the pores is usually less than about 2 nm.

As used herein, the term “fuel-grade” with regard to an ethanol solution refers to ethanol which is suitable for use as a fuel, either instead of or in combination with gasoline. In order to be suitable for use as a fuel, ethanol must have a low water content (preferably less than about 3.5% (v/v) water, more preferably less than 1%).

As used herein, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein. Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed. As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

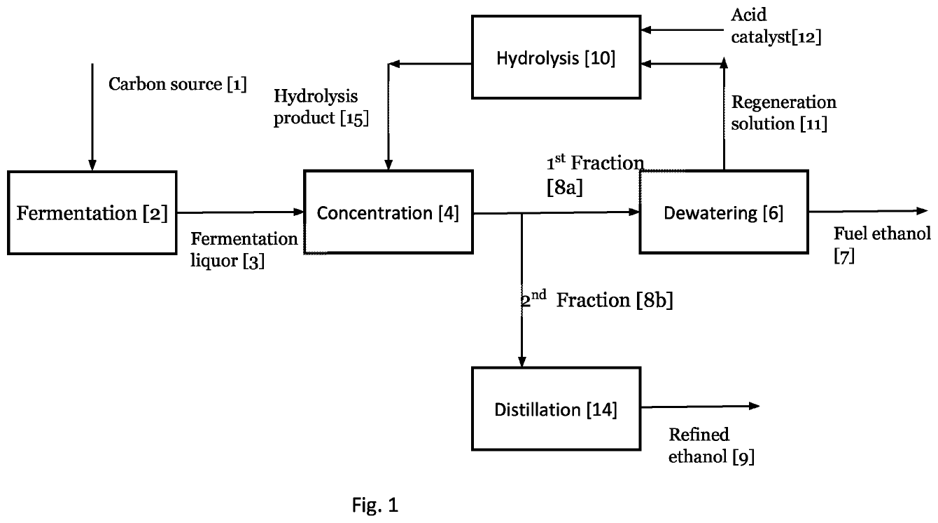

Referring now to FIG. 1, there is shown a schematic representation of an embodiment of a method of producing low-acetal ethanol, in accordance with the principles of the present invention.

According to the method, a carbon source 1 is fermented with an ethanol-producing organism in a fermentation step 2 to provide a fermentation liquor 3. Fermentation liquor 3 comprises about 15% wt ethanol and about 85% wt at water.

Fermentation liquor 3 is concentrated in step 4 to forma concentrated ethanol solution containing between 85% wt and 96% wt ethanol and between 4% wt and 15% wt of water. The concentrated ethanol solution is divided into a first fraction 8a and a second fraction 8b. First fraction 8a is then dewatered in step 6, such as by use of a molecular sieve, to produce fuel-grade ethanol 7. Second fraction 8b is distilled in step 14 to provide refined ethanol 9.

The molecular sieve is regenerated to desorb water which has been absorbed, thereby producing regeneration solution 11. Regeneration solution 11 is contacted with an acid catalyst 12 and hydrolyzed in step 1o to provide hydrolysis product 15. Hydrolysis product 15 is recycled to said fermentation liquor prior to said or during said concentration 4.

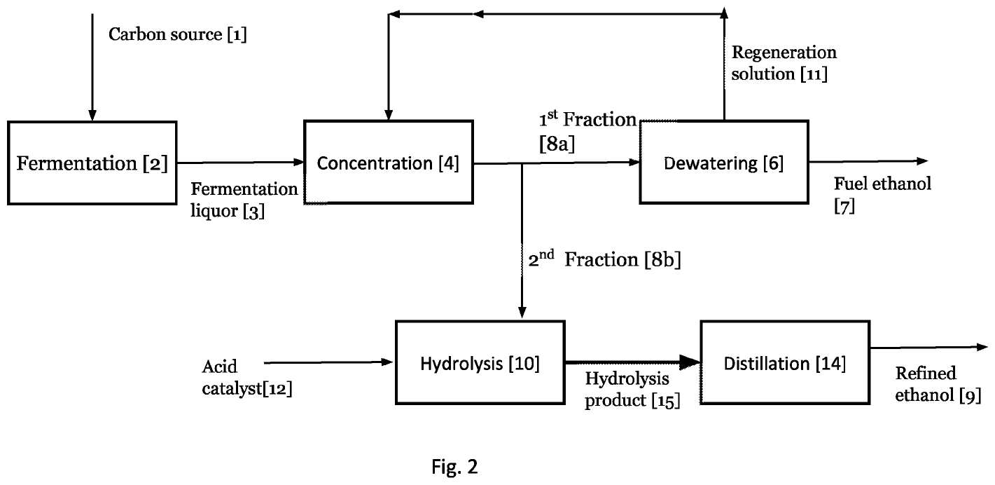

Referring now to FIG. 2, there is shown a schematic representation of an alternative embodiment of a method of producing low-acetal ethanol, in accordance with the principles of the present invention.

According to the method, a carbon source 1 is fermented with an ethanol-producing organism in a fermentation step 2 to provide a fermentation liquor 3. Fermentation liquor 3 comprises about 15% wt ethanol and about 85% wt water.

Fermentation liquor 3 is concentrated in step 4 to form a concentrated ethanol solution containing between 85% wt and 96% wt ethanol and between 4% wt and 15% wt of water. The concentrated ethanol solution is divided into a first fraction 8a and a second fraction 8b. First fraction 8a is then dewatered in step 6, such as by use of a molecular sieve, to produce fuel-grade ethanol 7.

The molecular sieve is regenerated to desorb water which has been absorbed, thereby producing regeneration solution 11. Regeneration solution 11 is recycled to said fermentation liquor prior to or during said concentration 4.

Second fraction 8b is contacted with an acid catalyst 12 and hydrolyzed in step 10 to provide hydrolysis product 15. Hydrolysis product 15 is distilled in step 14 to provide refined ethanol 9.

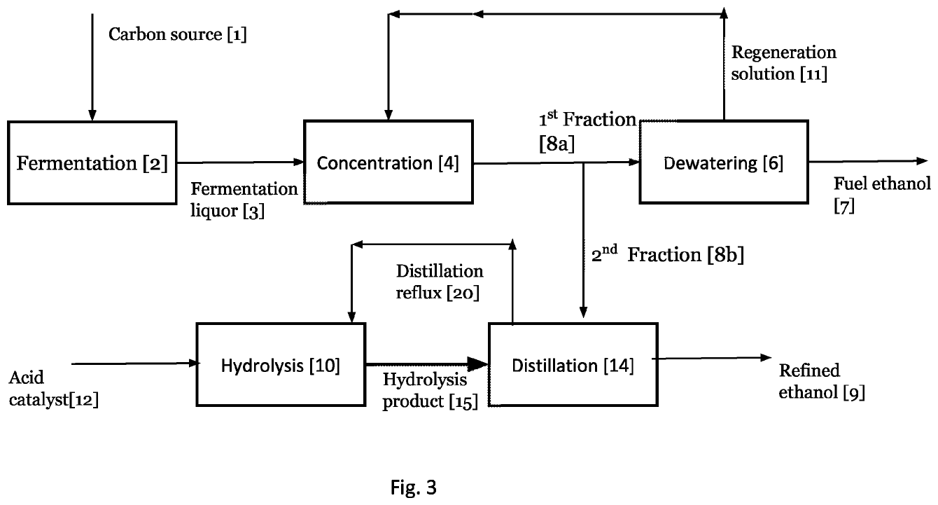

Referring now to FIG. 3, there is shown a schematic representation of a further alternative embodiment of a method of producing low-acetal ethanol, in accordance with the principles of the present invention.

According to the method, a carbon source 1 is fermented with an ethanol-producing organism in a fermentation step 2 to provide a fermentation liquor 3. Fermentation liquor 3 comprises about 15% wt ethanol and about 85% wt water.

Fermentation liquor 3 is concentrated in step 4 to forma concentrated ethanol solution containing between 85% wt and 96% wt ethanol and between 4% wt and 15% wt of water. The concentrated ethanol solution is divided into a first fraction 8a and a second fraction 8b. First fraction 8a is then dewatered in step 6, such as by use of a molecular sieve, to produce fuel-grade ethanol 7.

The molecular sieve is regenerated to desorb water which has been absorbed, thereby producing regeneration solution 11. Regeneration solution 11 is recycled to said fermentation liquor prior to or during said concentration 4.

Second fraction 8b is contacted with an acid catalyst 12 and hydrolyzed in step 10 to provide hydrolysis product 15. Hydrolysis product 15 is distilled in step 14 to provide refined ethanol 9 and a distillation reflux product 20. Distillation reflux product 20 is recycled to the hydrolysis reaction 10 and further distilled in step 14 to provide refined ethanol 9.

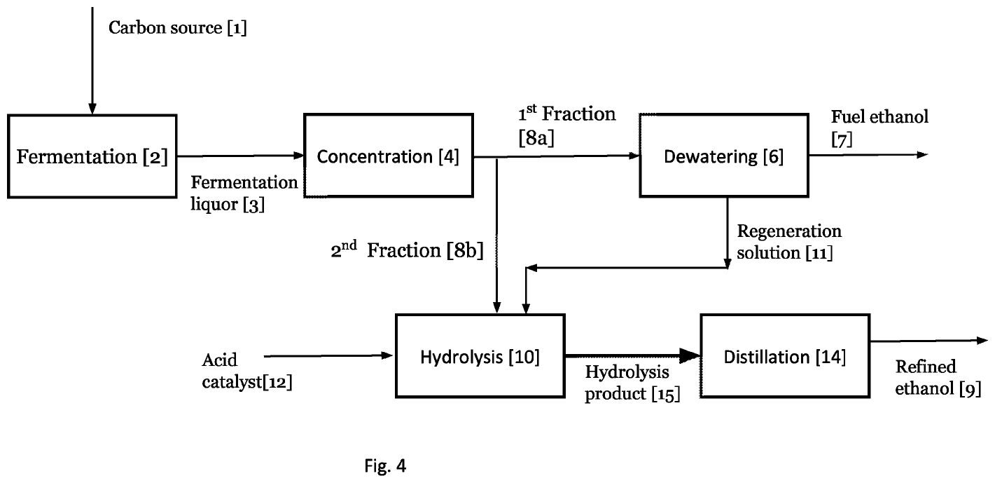

Referring now to FIG. 4, there is shown a schematic representation of a further alternative embodiment of a method of producing low-acetal ethanol, in accordance with the principles of the present invention.

According to the method, a carbon source 1 is fermented with an ethanol-producing organism in a fermentation step 2 to provide a fermentation liquor 3. Fermentation liquor 3 comprises about 15% wt ethanol and about 85% wt water.

Fermentation liquor 3 is concentrated in step 4 to form a concentrated ethanol solution containing between 85% wt and 96% wt ethanol and between 4% wt and 15% wt of water.

The concentrated ethanol solution is optionally divided into a first fraction 8a and a second fraction 8b, wherein first fraction 8a is then dewatered in step 6, such as by use of a molecular sieve, to produce fuel-grade ethanol 7. Alternatively, all of the concentrated ethanol solution is dewatered in step 6, such as by use of a molecular sieve, to produce fuel-grade ethanol 7 (not shown).

The molecular sieve is regenerated to desorb water which has been absorbed, thereby producing regeneration solution 11.

Regeneration solution 11 and second fraction 8b (when present) are contacted with an acid catalyst 12 and hydrolyzed in step 10 to provide hydrolysis product 15. Hydrolysis product 15 is distilled in step 14 to provide refined ethanol 9.

In some such embodiments, 100% of solution 11 is hydrolyzed to provide the refined ethanol product 9, which increases the proof of the molecular sieve feed and effectively increases the plant capacity.

### EXAMPLES

### Example 1: Hydrolysis of Acetal (1,1-Diethoxy Ethane) in Various Ethanol Proof Solution with Different Acid Catalysts

Ethanol solution at different proofs (100, 150, 190 or 200 proof) were provided and contacted with either nitric acid or sulfuric acid to provide 2 mM acid in the final solution.

Acetaldehyde, acetal and ethanol contents, pH, acetal/ethanol ratios and percentage hydrolysis was chromatographically determined for each solution after 1 hour and after 1.5 days of contact.

Results are presented below in Table 1 for measurements at 1 hour and in Table 2 for measurements at 1.5 days.

### Example 2: Hydrolysis of Acetal (1,1-Diethoxy Ethane) in 120 Proof Ethanol Solution with Various Concentrations of Nitric Acid Catalyst

120 proof ethanol solution was provided and contacted with nitric acid in an amount which provided 0.25 mM, 0.5 mM acid in the final solution.

Acetaldehyde, acetal and ethanol contents, acetal/ethanol ratios and percentage hydrolysis was determined for each solution after 1 hour of contact.

Results are presented below in Table 2.

TABLE 1

| | Proof (P)/acid | | | | | | | |
|---|---|---|---|---|---|---|---|---|
| | 200P/ | 200P/ | 190P/ | 190P/ | 150P/ | 150P/ | 100P/ | 100P/ |
| | H2SO₄ | HNO₃ | H2SO₄ | HNO₃ | H2SO₄ | HNO₃ | H2SO₄ | HNO₃ |
| Acid | 2 | 2 | 2 | 2 | 2 | 2 | 2 | 2 |
| concentration | | | | | | | | |
| (mM) | | | | | | | | |
| Acetaldehyde | ND | ND | 93 | 213 | 235 | 260 | 259 | 271 |
| (ppm) | | | | | | | | |
| Acetal (ppm) | 742 | 936 | 663 | 504 | 303 | 317 | 111 | 125 |
| Acetal/EtOH | 0.0074 | 0.0086 | 0.0066 | 0.0046 | 0.0030 | 0.0029 | 0.0011 | 0.0012 |
| Percent | 0.0% | 0.0% | 10.6% | 46.1% | 59.1% | 66.1% | 85.0% | 86.7% |
| hydrolysis | | | | | | | | |
| pH | 1.38 | 1.33 | 2.21 | 2.26 | 2.71 | 2.8 | 2.76 | 2.95 |

TABLE 2

| | Proof (P)/acid | | | | | | | |
|---|---|---|---|---|---|---|---|---|
| | 200P/ | 200P/ | 190P/ | 190P/ | 150P/ | 150P/ | 100P/ | 100P/ |
| | H2SO₄ | HNO₃ | H2SO₄ | HNO₃ | H2SO₄ | HNO₃ | H2SO₄ | HNO₃ |
| Acid | 0.25 | 0.25 | 0.25 | 0.25 | 0.5 | 0.5 | 1 | 1 |
| concentration | | | | | | | | |
| (mM) | | | | | | | | |
| Acetaldehyde | 101 | 145 | 160 | 167 | 255 | 221 | 281 | 275 |
| (ppm) | | | | | | | | |
| Acetal (ppm) | 635 | 556 | 489 | 437 | 291 | 250 | 209 | 204 |
| Acetal/EtOH | 0.0059 | 0.0051 | 0.0045 | 0.0040 | 0.0027 | 0.0023 | 0.0019 | 0.0019 |
| Percent | 32.2% | 40.6% | 47.7% | 53.3% | 68.9% | 73.3% | 77.7% | 78.2% |
| hydrolysis | | | | | | | | |

1. (canceled)

2. (canceled)

3. (canceled)

4. (canceled)

5. (canceled)

6. A method for the production of refined, low-acetal ethanol, comprising
(i) providing a feed stream comprising between 30% wt and 99% wt ethanol, between 70% wt and 1% wt water and between 20 and 2000 parts per million acetal;
(ii) contacting said feed stream with an acid catalyst, whereby at least 20% of the acetal hydrolyses to form a hydrolysis product comprising ethanol, water, acetaldehyde and optionally residual acetal; and
(iii) distilling at least a fraction of said hydrolysis product, whereby refined, low-acetal ethanol is formed.

7. The method of claim 6, wherein said providing said feed stream comprises:
(i) providing a carbon source;
(ii) fermenting said carbon source to form a fermentation liquor;
(iii) concentrating a fraction of said fermentation liquor to form a Concentrated ethanol solution containing between 85% wt and 96% wt ethanol and between 4% wt and 15% wt of water;
(iv) dewatering a first fraction of said concentrated ethanol solution on a molecular sieve to form a fuel-grade, dewatered ethanol solution containing less than 1% wt water, wherein said first fraction comprises between 10% wt and 95% wt of the ethanol in the concentrated ethanol solution; and
(v) regenerating said molecular sieve, whereby a feed stream comprising a regeneration solution is formed, which feed stream comprises between 30% wt and 99% wt ethanol, between 70% wt and 1% wt water and between 20 and 2000 parts per million acetal;
wherein the method further comprises recycling said hydrolysis product to said fermentation liquor prior to or during said concentrating of said fermentation liquor; and
distilling a second fraction of said concentrated ethanol solution, comprising between 5% wt and 90% wt of the ethanol in the concentrated ethanol solution, whereby refined, low-acetal ethanol is formed.

8. The method of claim 6, wherein said providing said feed stream comprises
(i) providing a carbon source;
(ii) fermenting said carbon source to form a fermentation liquor;
(iii) concentrating at least a fraction of said fermentation liquor to form a concentrated ethanol solution containing between 85% wt and 96% wt ethanol and between 4% wt and 15% wt of water;
(iv) dewatering a first fraction of said concentrated ethanol solution on a molecular sieve to form a fuel-grade, dewatered ethanol solution containing less than 1% wt water, wherein said first fraction comprises between 10% wt and 95% wt of the ethanol in the concentrated ethanol solution;
(v) regenerating said molecular sieve, whereby a regeneration solution is formed, and
(vi) recycling said regeneration solution to said fermentation liquor prior to or during said concentrating of said fermentation liquor, whereby said feed stream is formed, which feed stream comprises between 30% wt and 99% wt ethanol, between 70% wt and 1% wt water and between 20 and 2000 parts per million acetal;
wherein said contacting said feed stream with said acid catalyst comprises contacting a fraction of said feed stream comprising between 5% wt and 90% wt of the ethanol in the feed stream with said acid catalyst.

9. The method of claim 1, wherein said providing said feed stream comprises:
(i) providing a carbon source;
(ii) fermenting said carbon source to form a fermentation liquor;
(iii) concentrating at least a fraction of said fermentation liquor to form a concentrated ethanol solution containing between 85% wt and 96% wt ethanol and between 4% wt and 15% wt of water;
(iv) dewatering a first fraction of said concentrated ethanol solution on a molecular sieve to form a fuel-grade, dewatered ethanol solution containing less than 1% wt water, wherein said first fraction comprises between 10% wt and 95% wt of the ethanol in the concentrated ethanol solution;
(v) regenerating said molecular sieve, whereby a regeneration solution is formed,
(vi) recycling said regeneration solution to said fermentation liquor prior to or during said concentrating of said fermentation liquor; and
(vii) distilling a second fraction of said concentrated ethanol solution comprising between 5% wt and 90% wt of the ethanol in said concentrated solution, whereby a feed stream comprising a fraction of the distilled ethanol is formed, which feed stream comprises between 30% wt and 99% wt ethanol, between 70% wt and 1% wt water and between 20 and 2000 parts per million acetal;
wherein said contacting said feed stream with said acid catalyst comprises contacting at least a fraction of said feed stream comprising between 5% wt and 90% wt of the ethanol in the feed stream with said acid catalyst,
wherein the method further comprises recycling said hydrolysis product to said distilling and/or distilling said hydrolysis product to a second distilling.

10. The method of claim 1, wherein said acid catalyst is selected from the group consisting of mineral acids, polymers carrying —SO₃ functions and acidic zeolites.
